(19) United States
(12) Patent Application Publication
(10) Pub. No.: US 2025/0312481 A1
(43) Pub. Date: Oct. 9, 2025

(54) GELATIN AND LIPIDOID GENETIC DELIVERY PARTICLE

(71) Applicant: The Curators of the University of Missouri, Columbia, MO (US)

(72) Inventors: Raghuraman Kannan, Columbia, MO (US); Abilash Gangula, Columbia, MO (US); Dhananjay Suresh, Columbia, MO (US); Agasthya Suresh Babu, Columbia, MO (US); Anandhi Upendran, Columbia, MO (US); Zhaohui Li, Columbia, MO (US)

(21) Appl. No.: 19/092,157

(22) Filed: Mar. 27, 2025

Related U.S. Application Data
(60) Provisional application No. 63/573,986, filed on Apr. 3, 2024.

(51) Int. Cl. A61K 47/69; A61K 9/51; A61K 47/68; C12N 9/22; C12N 15/113

(52) U.S. Cl. CPC A61K 47/6925; A61K 9/5123; A61K 9/5169; A61K 47/6835; C12N 9/22; C12N 15/113; C12N 2310/14; C12N 2310/20

(57) ABSTRACT

A genetic delivery nanoparticle includes gelatin, a lipidoid, and a genetic molecule payload. The genetic molecule payload can be an RNA, DNA or Crispr system payload. SiRNA is an example payload and can be encapsulated with the gelatin and lipidoid and covalently conjugated to a surface of the nanoparticle. The nanoparticle surface can also include an antibody and PEG conjugated to it. A method for forming genetic delivery nanoparticle includes forming an adduct of gelatin, the lipidoid and a genetic payload; and cross-linking the gelatin to form the genetic delivery nanoparticle. Varying the size of a carbon chain in the lipidoid controls the size of the formed genetic delivery nanoparticle.

### PRIORITY CLAIM AND REFERENCE TO RELATED APPLICATION

The application claims priority under 35 U.S.C. § 119 from prior U.S. provisional application Ser. No. 63/573,986, which was filed Apr. 3, 2024.

### FEDERALLY SPONSORED RESEARCH AND DEVELOPMENT

This invention was made with government support under CA274677 awarded by the National Institutes of Health. The government has certain rights in the invention.

### FIELD

Fields of the invention include nanoparticles and complex structures including nanoparticles, as well as sensitive labile molecule payloads, and particles for genetic material delivery.

### BACKGROUND

RNA interference (RNAi) has shown significant potential in cancer treatment by silencing oncogenes. For RNAi to be successful clinically, a carrier is required to deliver siRNA safely and efficiently to the target site. Researchers have developed liposomal or polymeric nanocarriers using natural and synthetic materials to protect siRNA from physiological barriers, enhance cellular uptake, and promote endosomal escape. See, Mansoori, B.; Sandoghchian Shotorbani, S.; Baradaran, B. RNA Interference and Its Role in Cancer Therapy. Adv. Pharm. Bull. EISSN 2251-7308 2014; Xin, Y.; Huang, M.; Guo, W. W.; Huang, Q.; Zhang, L. Z.; Jiang, G. Nano-Based Delivery of RNAi in Cancer Therapy. Mol. Cancer 2017, 16 (1), 134; Moazzam, M.; Zhang, M.; Hussain, A.; Yu, X.; Huang, J.; Huang, Y. The Landscape of NanoparticleBased siRNA Delivery and Therapeutic Development. Mol. Ther. 2024, 32

The use of liposomes is mainly limited to hepatic diseases due to their inherent affinity to ApoE receptors. Liposomes also tend to suffer from premature drug leakage during storage or systemic circulation due to the fluidity of the encapsulating lipid layer. See, e.g., Pasarin, D.; Ghizdareanu, A.-I.; Enascuta, C. E.; Matei, C. B.; Bilbie, C.; Paraschiv-Palada, L.; Veres, P. A. Coating Materials to Increase the Stability of Liposomes. Polymers 2023, 15 (3), 782.

Polymeric nanoparticles are another delivery vehicle. However, polymer nanoparticles suffer from burst release and render inadequate stability to encapsulated RNA. Kamaly, N.; Yameen, B.; Wu, J.; Farokhzad, O. C. Degradable Controlled-Release Polymers and Polymeric Nanoparticles: Mechanisms of Controlling Drug Release. Chem. Rev. 2016, 116 (4), 26022663; Lu, X.-Y.; Wu, D.-C.; Li, Z.-J.; Chen, G.-Q. Polymer Nanoparticles. In Progress in Molecular Biology and Translational Science; Elsevier, 2011; Vol. 104, pp 299-323; Hasan, A. S.; Socha, M.; Lamprecht, A.; Ghazouani, F. E.; Sapin, A.; Hoffman, M.; Maincent, P.; Ubrich, N. Effect of the Microencapsulation of Nanoparticles on the Reduction of Burst Release. Int. J. Pharm. 2007, 344 (1-2), 53-61.

Gelatin is biocompatible, biodegradable, and can evade reticuloendothelial system clearance (RES), and has been used in nanoparticle form to incorporated siRNA. Gelatin-based formulations are commonly used in clinics. Suresh, D.; Suresh, A.; Kannan, R. Engineering Biomolecular Systems: Controlling the Self-Assembly of Gelatin to Form Ultra-Small Bioactive Nanomaterials. Bioact. Mater. 2022, 18, 321-336; Ishikawa, H.; Nakamura, Y.; Jo, J.; Tabata, Y. Gelatin Nanospheres Incorporating siRNA for Controlled Intracellular Release. Biomaterials 2012, 33 (35), 9097-9104; Young, S.; Wong, M.; Tabata, Y.; Mikos, A. G. Gelatin as a Delivery Vehicle for the Controlled Release of Bioactive Molecules. J. Controlled Release 2005, 109 (1-3), 256-27; Babu, R. J.; Annaji, M.; Alsaqr, A.; Arnold, R. D. Animal-Based Materials in the Formulation of Nanocarriers for Anticancer Therapeutics. In Polymeric Nanoparticles as a Promising Tool for Anticancer Therapeutics; Elsevier, 2019; pp 319-34.

The gelatin matrix is superior to polymeric nanoparticles as it ensures a steady, sustained release of cargo without any sudden releases. In comparison to liposomes, they are generally more stable at different temperatures and pH levels. Additionally, modifying and functionalizing them is much simpler than liposomes due to the presence of numerous functional groups. Khramtsov, P.; Burdina, O.; Lazarev, S.; Novokshonova, A.; Bochkova, M.; Timganova, V.; Kiselkov, D.; Minin, A.; Zamorina, S.; Rayev, M. Modified Desolvation Method Enables Simple One-Step Synthesis of Gelatin Nanoparticles from Different Gelatin Types with Any Bloom Values. Pharmaceutics 2021, 13 (10), 1537; Sivera, M.; Kvitek, L.; Soukupova, J.; Panacek, A.; Prucek, R.; Vecerova, R.; Zboril, R. Silver Nanoparticles Modified by Gelatin with Extraordinary pH Stability and Long-Term Antibacterial Activity. PLOS ONE 2014, 9 (8), e 103675; Baydin, T.; Aarstad, O. A.; Dille, M. J.; Hattrem, M. N.; Draget, K. I. Long-Term Storage Stability of Type A and Type B Gelatin Gels: The Effect of Bloom Strength and Co-Solutes. Food Hydrocoll. 2022, 127, 107535; Feng. X.; Dai, H.; Ma, L.; Yu, Y.; Tang, M.; Li, Y.; Hu, W.; Liu, T.; Zhang, Y. Food-Grade Gelatin Nanoparticles: Preparation, Characterization, and Preliminary Application for Stabilizing Pickering Emulsions. Foods 2019, 8 (10), 479;

Few publications discuss gelatin nanoparticles (GNPs) for RNA delivery. Gelatin is a polyampholyte polymer carrying a mixture of positive and negative charges in its backbone. Goudie, K. J.; McCreath, S. J.; Parkinson, J. A.; Davidson, C. M.; Liggat. J. J. Investigation of the Influence of PH on the Properties and Morphology of Gelatin Hydrogels. J. Polym. Sci. 2023, 61 (19), 2316-2332. Unmodified GNPs lack adequate electrostatic charges to condense and stabilize the large negatively charged RNA material. Lee, S. J.; Yhee, J. Y.; Kim, S. H.; Kwon, I. C.; Kim, K. Biocompatible Gelatin Nanoparticles for TumorTargeted Delivery of Polymerized siRNA in Tumor-Bearing Mice. J. Controlled Release 2013, 172 (1), 358-366. The lack of electrostatic charge makes it difficult to load (or encapsulate) genetic material onto GNPs, Andrée, L.; Oude Egberink, R.; Dodemont, J.; Hassani Besheli, N.; Yang, F.; Brock, R.; Leeuwenburgh, S. C. G. Gelatin Nanoparticles for Complexation and Enhanced Cellular Delivery of mRNA. Nanomaterials 2022, 12 (19), 3423. These publications only contemplate attaching to RNA to the surface of the GNPs and reported limited success.

Work by some of the present inventors successfully covalently attached RNA to the GNP surface. This showed limited success. Suresh, D.; Zambre, A.; Mukherjee, S.; Ghoshdastidar, S.; Jiang. Y.; Joshi, T.; Upendran, A.; Kannan, R. Silencing AXL by Covalent siRNA-Gelatin-Antibody Nanoconjugate Inactivates mTOR/EMT Pathway and Stimulates P53 for TKI Sensitization in NSCLC. Nanomedicine Nanotechnol. Biol. Med. 2019, 20, 102007; Srikar, R.; Suresh, D.; Zambre, A.; Taylor, K.; Chapman, S.; Leevy, M.; Upendran, A.; Kannan, R. Targeted Nanoconjugate Co-Delivering siRNA and Tyrosine Kinase Inhibitor to KRAS Mutant NSCLC Dissociates GAB1-SHP2 Post Oncogene Knockdown. Sci. Rep. 2016, 6 (1), 30245. These GNPs only included RNA on the surface.

Researchers have used several synthetic and natural material to design nanoparticle delivery vehicles. The conventional approach has improved gelatin's gene silencing efficiency by covalently modifying it with an array of cationic molecules, including spermine, 1,2-Ethanediamine, and polyethyleneimine.

Other publications are discussed individually below.
Zhang, W.; Han, B.; Lai, X.; Xiao, C.; Xu, S.; Meng. X.; Li, Z.; Meng. J.; Wen, T.; Yang, X.; Liu, J.; Xu, H. Stiffness of Cationized Gelatin Nanoparticles Is a Key Factor Determining RNAi Efficiency in Myeloid Leukemia Cells. Chem. Commun. 2020, 56 (8). The publication discusses cationized gelatin with ethylenediamine to form cationic gelatin NPs that can electrostatically bind to siRNA. The zeta potential of these particles is highly positive. Cationic particles are known to cause multifaceted toxicity making them unsuitable for clinical applications. By modifying the gelatin, these methods produce gelatin derivatives that are different from its natural biocompatible form and can introduce safety concerns. Upon exposure to plasma, the unshielded siRNA on the surface may be displaceable by other highly charged molecules or degraded by nucleases.
Mimi, H.; Ho, K. M.; Siu, Y. S.; Wu, A.; Li, P. Polyethyleneimine-Based Core-Shell Nanogels: A Promising siRNA Carrier for Argininosuccinate Synthetase mRNA Knockdown in HeLa Cells. J. Controlled Release 2012, 158 (1), 123-130 This publication discusses cationic molecule polyethyleneimine (PEI) to cationize gelatin and use of the cationized gelatin to form highly positive gelatin NPs that can bind with RNA. The drawbacks remain the same as that of as discussed in the previous paragraph with respect to Zhang et al.
Zhao, X.; Xi, Y.; Zhang, Y.; Wu, Q.; Meng, R.; Zheng, B.; Rei, L. Redox-Sensitive Gelatin/Silica-Aptamer Nanogels for Targeted siRNA Delivery. Nanoscale Res. Lett. 2019, 14 (1), 273. This publication discusses covalently conjugated siRNA to Gelatin NP using a disulfide linkage. Gelatin was modified with 3-aminopropyl trimethoxy silane to be able to generate functional groups for covalent conjugation of siRNA. As a result, these NPs are positively charged and raises toxicity concerns as discussed above.
Xu, J.; Ganesh, S.; Amiji, M. Non-Condensing Polymeric Nanoparticles for Targeted Gene and siRNA Delivery. Int. J. Pharm. 2012, 427 (1), 21-34. This publication discusses the use of polymeric nanoparticles such as gelatin to encapsulate RNA without cationizing so that the toxicity can be avoided. Relatively poor downregulation efficiency is provided.
Hajj, K. A.; Ball, R. L.; Deluty, S. B.; Singh, S. R.; Strelkova, D.; Knapp, C. M.; Whitehead, K. A. Branched-Tail Lipid Nanoparticles Potently Deliver mRNA In Vivo Due to Enhanced Ionization at Endosomal pH. Small 2019, 15 (6), 1805097. This publication also discusses a cationic lipid for gene deliver. The drawbacks of cationic lipids with positive charges is discussed above.
Whitehead, K. A.; Sahay, G.; Li, G. Z.; Love, K. T.; Alabi, C. A.; Ma, M.; Zurenko, C.; Querbes, W.; Langer, R. S.; Anderson, D. G. Synergistic Silencing: Combinations of Lipid-like Materials for Efficacious siRNA Delivery. Mol. Ther. 2011, 19 (9), 1688-1694. This publication also discusses a cationic lipid for gene deliver. The drawbacks of cationic lipids with positive charges is discussed above.
Madkhali, O. A. Drug Delivery of Gelatin Nanoparticles as a Biodegradable Polymer for the Treatment of Infectious Diseases: Perspectives and Challenges. Polymers 2023, 15 (21), 4327. This publication discusses the delivery of drugs, and does not provide a particle or method for delivery of genetic material.
Fan, Y.; Dhaliwal, H. K.; Menon, A. V.; Chang, J.; Choi, J. E.; Amiji, M. M.; Kim, J. Site-Specific Intestinal DMT1 Silencing to Mitigate Iron Absorption Using pH-Sensitive Multi-Compartmental Nanoparticulate Oral Delivery System. Nanomedicine Nanotechnol. Biol. Med. 2019, 22, 102091. This publication a discusses the use of polymeric nanoparticles such as gelatin to encapsulate RNA without cationizing so that the toxicity can be avoided. Relatively poor downregulation efficiency is provided.
Attarwala, H.; Clausen, V.; Chaturvedi, P.; Amiji, M. M. Cosilencing Intestinal Transglutaminase-2 and Interleukin-15 Using Gelatin-Based Nanoparticles in an in Vitro Model of Celiac Disease. Mol. Pharm. 2017, 14 (9), 3036-3044. This publication discusses the use of polymeric nanoparticles such as gelatin to encapsulate RNA without cationizing so that the toxicity can be avoided. Relatively poor downregulation efficiency is provided.

### SUMMARY

A preferred genetic delivery nanoparticle includes gelatin, a lipidoid, and a genetic molecule payload. The genetic molecule payload can be an RNA, DNA or Crispr system payload. SiRNA is an example payload and can be encapsulated with the gelatin and lipidoid and covalently conjugated to a surface of the nanoparticle. The nanoparticle surface can also include an antibody and PEG conjugated to it. A method for forming genetic delivery nanoparticle includes forming an adduct of gelatin, the lipidoid and a genetic payload; and cross-linking the gelatin to form the genetic delivery nanoparticle. Varying the size of a carbon chain in the lipidoid controls the size of the formed genetic delivery nanoparticle.

### BRIEF DESCRIPTION OF THE DRAWINGS

FIG. 1 is a schematic diagram of a genetic delivery nanoparticle according to a preferred embodiment of the invention.

FIGS. 2A and 2B are schematic diagrams showing a preferred method for synthesizing the genetic delivery nanoparticle of FIG. 1.

FIG. 3 is experimental data shows that the cationic nature of genetic delivery nanoparticles increases the cytotoxicity of the nanoparticles.

### DETAILED DESCRIPTION OF THE PREFERRED EMBODIMENTS

A preferred embodiment genetic delivery nanoparticle includes gelatin, a lipidoid, and a genetic molecule payload. The genetic molecule payload can be an RNA, DNA or Crispr system payload. SiRNA is an example payload and can be encapsulated with the gelatin and lipidoid and covalently conjugated to a surface of the nanoparticle. The nanoparticle surface can also include an antibody and PEG conjugated to it.

A genetic delivery nanoparticle of an embodiment combines gelatin, lipidoids, and a genetic molecule payload in a ratio to provide a delivery system that is effective in gene silencing, biocompatible and imparts high stability to the genetic molecule payload. The nanoparticle encapsulates a genetic molecule-lipidoid complex within the gelatin matrix and retains an engineerable surface, to which various attachments can be conjugated. As an example, experiments shows that the nanoparticle surface could be covalently attached to amine containing PEG and Antibody, and thiol-containing siRNA. Experiments shows that siRNA molecules are attached in two different binding modes: electrostatic encapsulation and covalent conjugation to the surface. This allows different siRNAs in as payloads on a single nanoparticle via two different binding modes.

The present genetic delivery nanoparticles were tested in an experiment and showed that 40 micrograms of gelatin and less than 1.2 micrograms of siRNA carried via genetic delivery nanoparticle of a preferred embodiment, over 90% knockdown of target proteins in cancer cells (1 million) was achieved. With such a unique composition with multiple binding opportunities, the present genetic delivery nanoparticle provides a new approach for RNA delivery. Efficacy was demonstrated in cancer cells and animal models. Experiments shows that the present genetic delivery nanoparticles are non-toxic in mice models.

As siRNAs are inherently negative in charge, encapsulation or covalent conjugation would result in poor loading and target gene knockdown efficiency. To overcome this issue, a preferred method of nanoparticle synthesis uses cationic lipids as condensing molecules for RNA, and wraps the charged adducts with gelatin strands to form a hybrid nanoparticle. Gelatin is used in an amount required to nullify the positive charge while still providing availability for covalent attachment of siRNA on the surface. With a proper amount of gelatin, the positively charged “lipidoid: siRNA” adduct is shielded, and the native “negative” charge of gelatin prevails. To further enhance the efficiency of gene knockdown, siRNA can also be attached to the surface of the nanoparticles using thiol linkages. Preferred genetic delivery nanoparticles provide a negative zeta potential, unlike previously known gelatin-based siRNA carriers with a positive zeta potential. This means they are less toxic and better tolerated by cells, making them safer for therapeutic applications. Moreover, the dual mode of siRNA attachment provides unprecedented gene knockdown efficacy for gelatin nanoconstructs, which is expected to significantly improve the effectiveness of RNA-based therapies.

The present genetic delivery nanoparticles and present synthesis methods provide numerous advantages. To preserve gelatin's natural properties, methods avoid cationizing the backbone. Instead, lipidoids load genetic molecule payload electrostatically and encapsulate payload within a uniform gelatin matrix. The negative zeta potential of the present genetic delivery nanoparticles, the genetic molecule payload is efficiently shielded within the gelatin matrix that protects it from direct exposure to serum proteins. The present genetic delivery nanoparticles are bivalent in nature—with dual (electrostatic and covalent) modes of conjugation of genetic molecule payload allowing for high and different types of loading. As an example, the bivalency of the nanoparticles coupled with efficient loading and protection of siRNA resulted in complete gene silencing (>95%) in experiments. The present genetic delivery nanoparticles are further functionalizable; for example, the nanoparticles can be attached with PEG and target antibody. These surface groups can be tuned to prevent particle aggregation in the serum, modulate immune clearance, and enhance the stability and pharmacokinetics of the nanoparticles. The size tunability of the present genetic delivery nanoparticles provides an essential tool for effective tumor penetration. The size of the nanoparticle can be controlled with the size of lipidoid used in the synthesis. Smaller lipidoids yield smaller nanoparticles than larger lipidoids. A Go-C16 lipidoid produced a larger nanoparticle, while GoC8-resulted the size to <120 nm, which is a remarkable tuning ability. Go-C16, Go-C14 and Go-C8 lipidoids produce nanoparticles of successively smaller sizes with the Go-C16 being the largest and the Go-C8 lipidoid being the smallest size.

Preferred genetic delivery nanoparticles provide dual siRNA conjugation modes: i) covalent conjugation of siRNA on NP surface and ii) encapsulation of siRNA inside the nanoparticle using a cationic lipidoid. The cationic lipidoid plays a vital role in achieving higher downregulation efficiencies of target gene when compared with the gelatin nanoparticles with no lipidoid. This can be attributed to high siRNA loading, and efficient cytoplasmic release by promoting lysosomal escape. Bivalency or dual siRNA conjugation modes helps in higher siRNA loading and higher gene silencing efficacy. Overall, the present genetic delivery nanoparticles demonstrate superior downregulation efficiency compared to prior gelatin particles that only conjugated siRNA to the surface of the particles.

Preferred embodiments of the invention will now be discussed with respect to experiments and drawings. Broader aspects of the invention will be understood by artisans in view of the general knowledge in the art and the description of the experiments that follows.

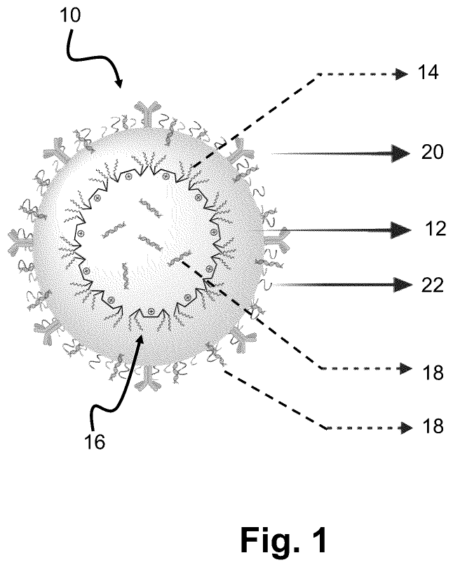

FIG. 1 shows genetic delivery nanoparticle 10 that is based upon a gelatin 12 and lipidoid 14 matrix 16. A genetic molecule payload 18 is encapsulated in the matrix 16. The genetic molecule payload can be an RNA, DNA or Crispr system payload. The payload 18 can be both electrostatically encapsulated within the gelatin 12 and lipidoid 14 and covalently conjugated to a surface of the nanoparticle 10. An antibody 20 can be conjugated to the surface of the nanoparticle 10. Additionally, PEG 22 can be conjugated to the surface of the nanoparticle 10.

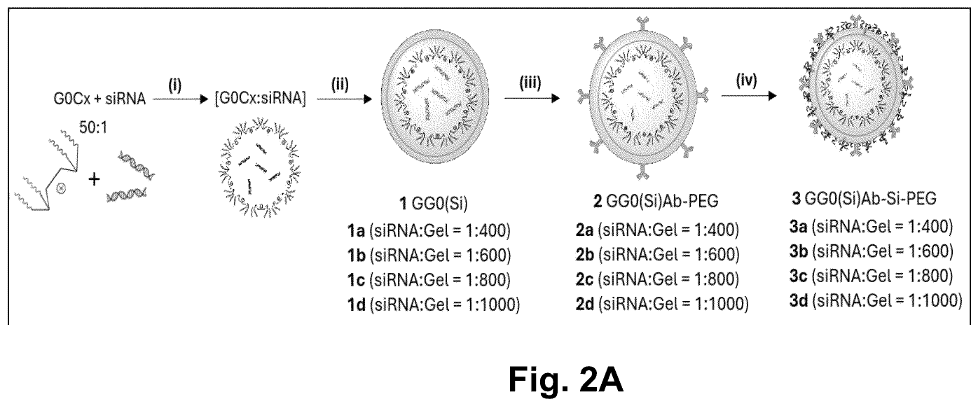

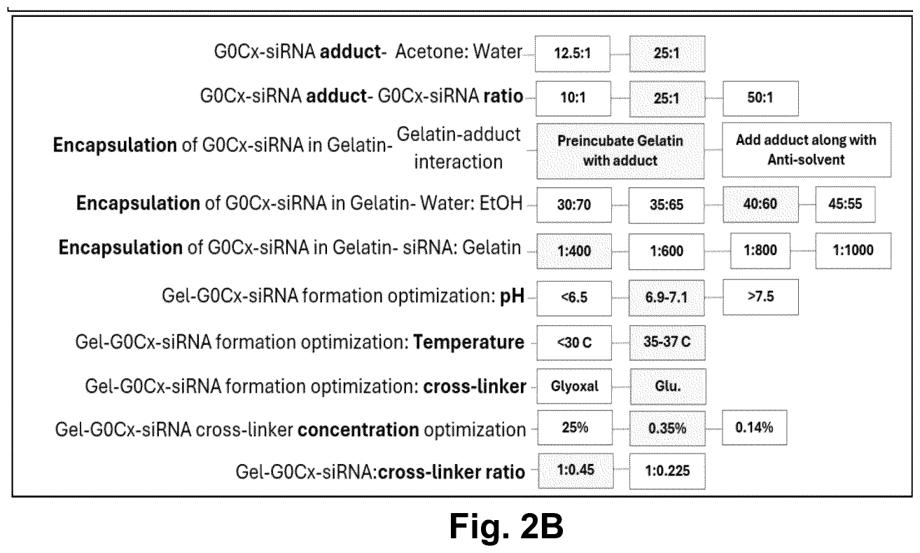

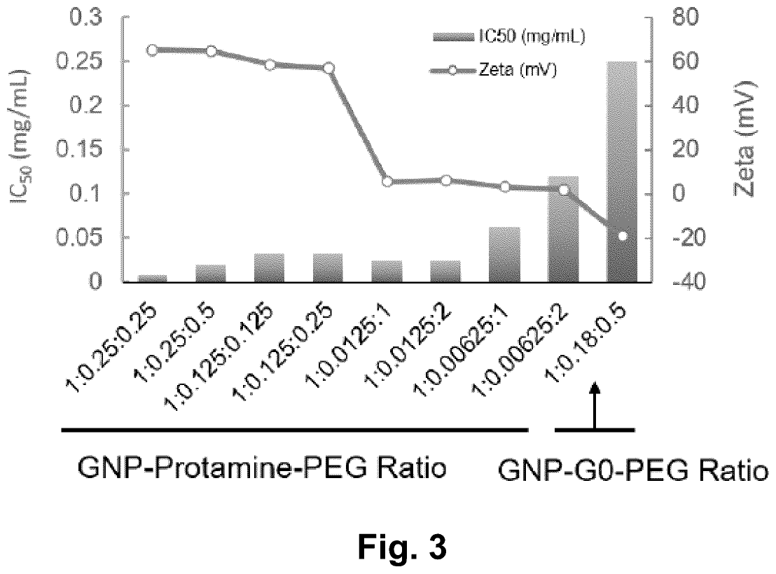

FIGS. 2A and 2B are schematic diagrams showing a preferred method for synthesizing the genetic delivery nanoparticle 10. FIG. 2A shows preferred fabrication steps. The first step involves forming an adduct that yields the genetic delivery nanoparticle upon interaction with gelatin. In a preferred example, the ratios in the first and second steps (i) Acetone:water (25:1); (ii) Gelatin (diff. ratio); Water:EtOH (40:60); pH 6.9-7.1; 35-37 C. The weight ratio of lipidoid to siRNA was 25:1 In step three, the genetic delivery nanoparticle are converted into a rigid structure and then functionalized with EGFR-antibody and PEG-2000. In a preferred example, the ratios in the third step and a fourth step to conjugate molecule to the surface: iii) Gelatin (different ratios), Glutaraldehyde 1:0.45 (0.35%); EDC/NHS, Ab, NH₂-PEG-2000; (iv) SMCC, HS-siRNA. The final genetic delivery nanoparticle is obtained by repeated washing and centrifugation. FIG. 2B illustrates several reactions to optimize the conditions for obtaining the genetic delivery nanoparticle 10. The shaded region represents the optimal condition that provided the highest yields of genetic delivery nanoparticles. The non-shaded conditions either failed to produce the product or resulted in intractable aggregates.

Experiments varied the amount of gelatin while keeping the amount of lipidoid-siRNA constant. The different weight ratios of siRNA to Gelatin that were investigated are 1:400, 1:600, 1:800; 1:1000. In terms of lipidoid, these ratios (lipidoid to gelatin weight ratio) translate to 25:400, 25:600, 25:800, 25:1000. All of these ratios successfully encapsulated siRNA in the genetic deliver nanoparticle.

A preferred stepwise assembly protocol was used to optimize the process. A first step involved enhancing the interaction between lipidoids and siRNA using an acetone-water solvent system. Solvent mixtures play an important role in stabilizing the electrostatic adduct between these two ions. A 25:1 acetone/water ratio (v/v) yielded an adduct with extended stability and less aggregation. Lower acetone concentrations resulted in an electrostatic adduct with poor solubility, causing them to be immiscible and intractable. Acetone/water ratios (v/v)≤12.5:1 are not recommended as it leads to precipitation of lipidoid-siRNA adduct. An acceptable range of acetone: water (v/v) is 17.5:1-25:1. Lipidoid amounts were also varied in relation to constant amount of siRNA. Interestingly, we found that a ratio of 25:1 (w/w) of lipidoid to siRNA was adequate to form a stable electrostatic adduct resulting in good gene silencing efficacy. An acceptable ratio range is 10:1 to 50:1. The lower ratios can sometimes result in lower downregulation efficiencies, while the higher ratios might contribute to toxicity. The optimum ratio is 25:1. The electrostatic adduct “G0Cx-Si” (x=8 or 14) showed a positive zeta potential, indicating that siRNA is enclosed within the cavity formed by the lipidoids. The adduct is stable only for a few minutes; leaving it for more than five minutes would result in adhering to the reaction vessel and render itself unusable. All admissible chemical treatments failed to bring them back to solution once settled.

The next step involves shielding the electrostatic adduct with gelatin coating with the simultaneous formation of the genetic delivery nanoparticles. It is likely that gelatin strands form layers around the cluster of adducts, which results in the spherical genetic delivery nanoparticle. It is expected that there will not be a selective number of clusters of adduct per particle since the process is based on physical diffusion combined with particle encapsulation. One goal in tuning fabrication is to minimize gelatin and maximize the target protein downregulation efficacy, and this goal can be achieved by determining the optimum amount of gelatin strands around the cluster of adducts. A first step is to trigger the formation of gelatin coating around the adduct. Experiments show that the ratio of 40:60 (v/v) yielded optimum results in the formation of genetic delivery nanoparticles. An acceptable water to ethanol ratio (v/v) range to is 47.5:52.5 to 37.5:62.5. The pH of the reaction system played an important role in stabilizing the final construct. Acidic pH failed to form uniform spherical layers around the adduct. Basic pH resulted in poor downregulation efficiency. The result was surprising given that the gelatin's negative charge would increase at basic pH, resulting in enhanced interaction with positively charged electrostatic adduct. The results confirm that at near neutral pH (˜6.8-7.0) with no additional charges on the gelatin strands, the encapsulation is mediated by statistical diffusion; after obtaining the optimal balance of hydrophobic charges, the gelatin layer yields spherical genetic delivery nanoparticles. A temperature range of 35-37 C is optimum, as a lower temperature than this resulted in quick gelation.

As the strands of gelatin layers are loosely bound, a cross-linking agent is important to bind these layers together and impart rigidity. We found that glutaraldehyde serves as a better cross-linking agent than glyoxal. Additionally, with a gelatin: glutaraldehyde weight ratio of 1:0.45, relatively rigid genetic delivery nanoparticles were obtained with high reproducibility. An acceptable range is 1:0.38 to 1:0.52.

The optimum amount of gelatin strands around the cluster of adducts can be determined. To reduce the presence of gelatin strands around the adduct while maintaining stability, experiments varied the amounts of gelatin while keeping the “GOCxSi” concentration constant, we changed the gelatin weight ratio from 400-1000 with increments of 200 with respect to siRNA. The final genetic delivery nanoparticles were purified and characterized. Labels applied to the coated cores were particles 1a-1d, while 2a-2d represented the same genetic delivery nanoparticles ut with the addition of Ab and PEG. Lastly, the dual siRNA genetic delivery nanoparticles were labeled as 3a-3d.

The conjugates 1-3 contain G0C14 as the lipidoid component. Using the optimized parameters, conjugates with G0C8 as the lipidoid component were also synthesized. G0C8 forms a compact electrostatic adduct, decreasing the overall size. The synthetic parameters are able to produce a range of genetic delivery nanoparticles with varying physical and chemical characteristics.

### Experimental Synthesis Details

An aqueous solution of gelation (10 mg/mL) was prepared by dissolving 50 mg of Type B Gelatin (Bloom strength 225) in 5 mL of ultra-pure water at 37° C., 750 rpm. In another vial, lipidoid-siRNA complex was formed by smoothly mixing 500 μL of G0-C14 (2.5 mg/mL in acetone) with 20 μL of siAXL (200 μM) and incubating at room temperature for 1 min. An example of smooth mixing, lipidoid solution was added to a vial containing siRNA solution using a pipette at a rate of 2 drops per sec. After addition, the mixture was mixed using a pipette by repeating slow aspiration and dispense cycle for 6 times. To the gelatin solution, the lipidoidsiRNA complex was added dropwise at 37° C., 750 rpm. The pH of the mixture was adjusted to 7.0 using 0.2 N NaOH. To this mixture, around 8 ml of warm ethanol was added dropwise at 37° C., 750 rpm to trigger the formation of nanoparticles. The formation of nanoparticles was indicated by the appearance of turbidity. After 15 min, the nanoparticles were crosslinked by dropwise addition of 4 ml of the ethanol-water mixture (60% ethanol) containing 48 μL of glutaraldehyde (25% aqueous solution). The heat was turned off at this point. After an overnight reaction at 25° C., 750 rpm, the excess glutaraldehyde was quenched by adding 5 μL of 1 M glycine to the reaction mixture. The nanoparticles were washed with water by 5 cycles of centrifugation (15000 g for 8 min) and redispersion (35 mL of water). The washed pellet was dispersed in 250 μL of water to obtain Genetic delivery nanoparticles. The above is the optimized procedure for synthesizing Genetic delivery nanoparticles (1d) where the siRNA to gelatin ratio is 1:1000. Most of the procedure remains the same for synthesizing 1a, 1b, 1c, except that the initial amounts of gelatin taken for preparing 10 mg/ml solution are 20 mg, 30 mg, and 40 mg respectively. The volumes of 25% glutaraldehyde used for crosslinking 1a, 1b, 1c are 19.2 μL, 28.8 μL, 38.4 μL respectively. Similarly, volumes of 1 M glycine used for 1a, 1b, 1c are 2 μL, 3 μL, 4 μL respectively.

### Surface Conjugation of Antibody and PEG to Genetic Delivery Nanoparticles

Genetic delivery nanoparticles were conjugated to cetuximab antibody and NH2-PEG-COOH (2K daltons) by activation with EDC and sulfo-NHS mixture. Briefly, 8 mg of Genetic delivery nanoparticles was dispersed in 500 μL of MES buffer (0.1M, pH4.5). To this dispersion, 200 μL of MES buffer containing 1.52 mg of EDC and 1.76 mg of sulfo-NHS was added. The reaction mixture was incubated at 25° C., 800 rpm for 90 min followed by centrifugation at 15000 g for 6 min to obtain a pellet of activated Genetic delivery nanoparticles. The pellet was redispersed in 500 μL of 1×PBS and mixed with 1.2 mg of cetuximab antibody and 4 mg of NH2-PEG-COOH and the pH was adjusted to 7.2. The reaction mixture was incubated at 25° C., 800 rpm for 16 hours followed by centrifugation at 15000 g for 8 min. The pellet obtained was washed once with 1.5 mL of water and dispersed in 200 μL of water to obtain dispersion of Genetic delivery nanoparticles-Ab-PEG.

Surface Conjugation of siRNA to Genetic Delivery Nanoparticles

G(siRNA)-Ab-PEG NP serve as control for Genetic delivery nanoparticles and are synthesized using a similar protocol used for Genetic delivery nanoparticles except that siRNA is encapsulated without the help of lipidoid. Briefly, 50 mg of Type B Gelatin (Bloom strength 225) was dissolved in 5 mL of ultra-pure water at 37° C., 750 rpm. To this solution, 20 μL of 200 μM siAXL was added and the pH of the mixture was adjusted to 7.0. To this mixture, around 9.5 ml of warm ethanol was added dropwise at 37° C., 750 rpm to trigger the formation of nanoparticles. The nanoparticles were crosslinked by dropwise addition of 4 ml of the ethanol-water mixture (65% ethanol) containing 48 μL of glutaraldehyde (25% aqueous solution). After an overnight reaction at 25° C., 750 rpm, the excess glutaraldehyde was quenched by adding 5 μL of 1 M glycine to the reaction mixture. The nanoparticles were washed with water by 5 cycles of centrifugation (15000 g for 8 min) and redispersion (35 mL of water). Th protocol for conjugating antibody and PEG to these nanoparticles is same as the one used for Genetic delivery nanoparticles.

### Synthesis of G-Ab-PEG-siRNA NP.

G-Ab-PEG-siRNA NP serve as another control for Genetic delivery nanoparticles. The first step involves the synthesis of empty gelatin nanoparticles with no siRNA encapsulated within. The procedure for this step remains the same as the one used for synthesizing siRNA encapsulated gelatin nanoparticles in G (siRNA)-Ab-PEG except that siRNA is not added to the gelatin solution. In the next step, antibody and PEG are surface conjugated to the empty gelatin nanoparticles using the same procedure adopted for Genetic delivery nanoparticles. Finally, siRNA is surface conjugated to the nanoparticles using the same procedure followed for Genetic delivery nanoparticles.

### Synthesis of Cationized Gelatin Nanoparticles-siRNA Conjugated (GNP-Prot-PEG-siRNA).

The first step for synthesizing cationized gelatin nanoparticle is the synthesis of empty gelatin nanoparticles with no siRNA encapsulated within. The procedure for this step remains the same as the one used for synthesizing siRNA encapsulated gelatin nanoparticles in G (siRNA)-Ab-PEG except that siRNA is not added to the gelatin solution. The next step involves conjugation of protamine and antibody to the empty gelatin nanoparticles (GNP) using EDC, NHS chemistry. Briefly, 8 mg of GNP was dispersed in 500 μL of MES buffer (0.1M, pH4.5). To this dispersion, 200 μL of MES buffer containing 1.52 mg of EDC and 1.76 mg of sulfo-NHS was added. The reaction mixture was incubated at 25° C., 800 rpm for 90 min followed by centrifugation at 15000 g for 6 min to obtain a pellet of activated GNP. The pellet was redispersed in 500 μL of 1×PBS and mixed with protamine and NH2-PEG-COOK (2K). The pH was adjusted to 7.2. The reaction mixture was incubated at 25° C., 800 rpm for 16 hours followed by centrifugation at 15000 g for 8 min. The pellet obtained was washed once with 1.5 mL of water and dispersed in 200 μL of water to obtain dispersion of GNP-Prot-PEG. We have synthesized a library of GNP-Prot-PEG by varying the amounts of protamine and PEG reacted with 1 mg of activated GNP (GNP: Prot: PEG-1:0.25:0.5 to 1:0.00625:2). 20μ L of 200μ M siRNA was added to GNP-Prot-PEG dispersion obtained above and incubated overnight at 25° C., 800 rpm for 16 hours followed by centrifugation at 15000 g for 8 min. The pellet obtained was washed once with 1.5 mL of water and dispersed in 200 μL of water to obtain GNP-Prot-PEG-siRNA.

### Characterization

Genetic delivery nanoparticles were characterized using conventional analytical techniques, which include TEM and DLS. Genetic delivery nanoparticles are uniformly spherical and monodisperse with a size range of 220-260 nm. Interestingly, the size of Genetic delivery nanoparticles is relatively unaltered, whether or not the siRNA is present within the void. These findings suggest that the lipidoids create a “defined” space within the gelatin matrix, and the adduct formation with siRNA fits within the volume and retains the size. This data presents the rationale for the size decrease when we used a C8 lipidoid instead of C14. Indeed, by altering the C-length of the lipidoid, one can adjust the size of the Genetic delivery nanoparticles.

Zeta Potential: It is essential to determine the zeta potential of Genetic delivery nanoparticles (1-3) to understand the coating on the adduct “GO-Si”. It is worth noting that lipidoid has a very high positive zeta potential (>60 mV). However, upon complexation with siRNA, the zeta potential is reduced to +50 mV, which indicates that the siRNA is electrostatically bound to the lipidoid. To optimize the gelatin coating, we used different weight ratios of gelatin to siRNA and coated it on top of the adduct. The optimized genetic delivery nanoparticle construct showed a negative zeta potential (−15 mV), which suggests that the gelatin fibers effectively shielded the adduct and protected the positive charge from exposure to the surroundings. The study found that the zeta potential of Genetic delivery nanoparticles coated with a ratio of 1000:1 (1d, 2d, or 3d) does not differ from those synthesized from the ratio of 400:1 (1a, 2a, 3a). These results indicate that 1a with minimum amounts of gelatin strands evenly coats and effectively shields the electrostatic adduct. The implications of these findings are significant for the translational opportunities of Genetic delivery nanoparticles.

Interestingly, the negative charge on Genetic delivery nanoparticles remained after functionalization with Ab and PEG. The dual-siRNA Genetic delivery nanoparticles, wherein siRNA is also attached covalently to the surface showed minimal change in the zeta potential.

The lipidoid's high positive charge turns negative upon being coated with Gelatin. The negative charge of Genetic delivery nanoparticles indicates that the “GoSi” adduct is well-protected within the core of the nanoparticles

siRNA Loading: The siRNA load in the Genetic delivery nanoparticles (3a-3d) was estimated by using Cy5 labeled siRNA. In this study, we conjugated siRNA-Cy5 to Genetic delivery nanoparticles and estimated the amount present in the supernatant. This provides an indirect way to estimate the amount loaded within Genetic delivery nanoparticles. The standard curve of siRNA-Cy5 was used for the estimation. siRNA load was the highest for the bivalent gelatin nanoparticles compared to that of controls. The lesser the gelatin coating on the adduct, the siRNA loading per mg of the Genetic delivery nanoparticles would increase. While the 3d resulted in nanoparticles with siRNA load of ≈12.5 μg/mg, 3a resulted in significantly higher siRNA load of ≈28 μg/mg.

TABLE A

| Construct | siRNA Load | |
|---|---|---|
| G(Si) - Ab - PEG | ≈3 | μg/mg |
| G(GO) - Ab - PEG - Si | ≈3 | μg/mg |
| Genetic delivery nanoparticles (2 d) | ≈8.5 | μg/mg |
| Genetic delivery nanoparticles (3 d) | ≈12.5 | μg/mg |

TABLE B

| Construct | siRNA Load | |
|---|---|---|
| Genetic delivery nanoparticles (3a) | ≈28 | μg/mg |
| Genetic delivery nanoparticles (3b) | ≈19 | μg/mg |
| Genetic delivery nanoparticles (3c) | ≈17.5 | μg/mg |
| Genetic delivery nanoparticles (3d) | ≈12.5 | μg/mg |

siRNA loading in Genetic delivery nanoparticles estimated using Cy5 labeled siRNA: Table A shows a comparison of siRNA loading in dual siRNA Genetic delivery nanoparticles (3d) with single siRNA Genetic delivery nanoparticles (2d) and other controls. Table B shows a comparison of siRNA loading in dual siRNA Genetic delivery nanoparticles synthesized from increasing amount of gelatin (siRNA to gelatin ratio-1:400 to 1:1000). Genetic delivery nanoparticles synthesized from lowest amount of gelatin (3a, siRNA to gelatin ratio-1:400) showed the highest siRNA loading.

### Stability

Gene delivery vehicles need to protect siRNA from degradation by nucleases found in the serum before reaching the diseased site. The effectiveness of the nanocarrier depends on its ability to safeguard siRNA from degradation by serum nucleases. To evaluate the serum stability of siRNA associated with Genetic delivery nanoparticles, we used two different nanoconstructs representing bivalent Genetic delivery nanoparticles. In the first nanoconstruct, we used Genetic delivery nanoparticles where siRNA is electrostatically attached with G0-C14, and in the second, we attached siRNA covalently conjugated on the surface. We used siRNA-Cy5 for this experiment and evaluated stability using gel electrophoresis. In both cases, siRNA remained highly stable after incubating in the serum for 48 hours. Both the nanoparticles retained the siRNA intact for 24 to 48 hours. In the case of GelGO(Si), where the siRNA is electrostatically attached, it showed minor disintegration after 24 hours. In contrast, the covalent attachment of siRNA rendered very high stability, and no disintegration was observed for 48 hours. Combining both should provide a remarkable ability to retain the siRNA for longer than 48 hours. It is evident that Genetic delivery nanoparticles impart a high-level of stability to siRNA and protect it from serum nucleases.

### Cellular Toxicity

Cationic nanoparticles can pack high loads of siRNA due to strong electrostatic interactions and cause efficient downregulation of the target gene. Hence, the most common strategy adopted to enhance the siRNA loading and gene regulation efficiency of gelatin nanoparticles is to cationize them by modifications with positively charged molecules or polymers. However, the surface charge on the nanoparticles influences their cellular adherence, endocytosis, and toxicity. Literature suggests that use of cationic nanoparticles for cellular applications is limited by their toxicity arising from disruption of plasma-membrane integrity, damage to mitochondrial and lysosomes, and generation of large number of autophagosomes. Therefore, it is highly essential to shield the positive charge of nanoparticles used for siRNA delivery applications. To elucidate the difference in the toxicity between negatively charged Genetic delivery nanoparticles and regular cationic gelatin nanoparticles, we have synthesized protamine functionalized gelatin nanoparticle (GNP-Prot) which represents the cationized gelatin nanoparticle family and compared its toxicity with Genetic delivery nanoparticles. We evaluated the cellular toxicity using an MTT assay. In this study, we used a cancer cell line as the test system; the rationale for this choice is we will be using the same cell line in the next step to evaluate the target gene knockdown efficacy.

Protamine is a natural cationic peptide widely explored for RNA transfection. Hence, we chose protamine to covalently modify the surface of gelatin nanoparticles and cationize them. As expected, GNP-Prot efficiently transfected siRNA and downregulated the target gene at treatment concentration of 0.25 mg/mL. However, MTT assay showed that these particles are highly toxic to cells at significantly lower concentrations (IC50<0.008 mg/mL). Reducing the toxicity of these particles is essential to favor their use in therapeutic applications.

A library of GNP-Prot particles with reducing amounts of protamine and increasing amounts of PEG and evaluated their toxicity, as shown in the table below.

| Variants of | Amount of | Amount of | | Amount of | | | | | |
|---|---|---|---|---|---|---|---|---|---|
| GNP-Prot-PEG | Gel NP | Protamine | | NH2-PEG-OMe (2K) | | IC50 | | HDS | ZP |
| 1 | 1 mg | 0.25 | mg | 0.25 | mg | 0.008 | mg/ml | 332.1 nm | 65.3 mV |
| 2 | 1 mg | 0.25 | mg | 0.5 | mg | 0.021 | mg/ml | 330.2 nm | 64.8 mV |
| 3 | 1 mg | 0.125 | mg | 0.125 | mg | 0.0325 | mg/ml | 370.2 nm | 58.5 mV |
| 4 | 1 mg | 0.125 | mg | 0.25 | mg | 0.0325 | mg/ml | 380.1 nm | 56.8 mV |
| 5 | 1 mg | 0.0125 | mg | 1 | mg | 0.025 | mg/ml | 378.1 nm | 5.32 mV |
| 6 | 1 mg | 0.0125 | mg | 2 | mg | 0.025 | mg/ml | 345.5 nm | 6.02 mV |
| 7 | 1 mg | 0.00625 | mg | 1 | mg | 0.0625 | mg/ml | 364.5 nm | 3.05 mV |
| 8 | 1 mg | 0.00625 | mg | 2 | mg | 0.12 | mg/ml | 368.3 nm | 1.91 mV |

The rationale for adding PEG is based on the fact that PEGylation decreases the toxicity of particles. However, all the variants of GNP-Prot-PEG were found to be toxic to cells. Simultaneously, we evaluated whether cells can tolerate Genetic delivery nanoparticles in the concentration at which they can downregulate the target protein. The Genetic delivery nanoparticles were well-tolerated by cells, and very minimal cell death was observed (<20%), which is usually observed due to shock or stress. IC50 was not reached for the constructs at the highest concentration (0.25 mg/ml), suggesting the relative non-toxicity of the constructs. The presence of PEG played a role in reducing the cytotoxicity of Genetic delivery nanoparticles. GO-C14 encapsulation within Genetic delivery nanoparticles has not resulted in any increase in toxicity, suggesting the effective shielding of G0-C14 by nanoparticles.

Experiments showed that the genetic delivery nanoparticles are stable in serum. This allows the nanoparticles to be stored and transported. Another method for long-term storage is to to lyophilize the nanoparticles. A lyophilized powder is stable, allowing storage for an extended period without worrying about disintegration. A 60-day old Genetic delivery nanoparticle sample maintained the functionality of siRNA as indicated by its 90% downregulation efficiency.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A genetic delivery nanoparticle comprising gelatin, a lipidoid, and a genetic molecule payload.

2. The genetic delivery nanoparticle of claim 1, wherein the genetic molecule payload comprises an RNA, DNA or Crispr system payload.

3. The genetic delivery nanoparticle of claim 2, wherein the genetic molecule payload is siRNA.

4. The genetic delivery nanoparticle of claim 1, wherein the genetic molecule payload is electrostatically encapsulated with the gelatin and covalently conjugated to a surface of the nanoparticle.

5. The genetic delivery nanoparticle of claim 4, wherein the genetic molecule payload is siRNA.

6. The genetic delivery nanoparticle of claim 5, comprising an antibody conjugated to the surface of the nanoparticle.

7. The genetic delivery nanoparticle of claim 5, comprising PEG conjugated to the surface of the nanoparticle.

8. The genetic delivery nanoparticle of claim 1, wherein the lipidoid comprises Go-C14.

9. A genetic delivery nanoparticle consisting of gelatin, a lipidoid, a genetic molecule payload, and one or more materials attached a surface of the nanoparticle.

10. The genetic delivery nanoparticle of claim 9, wherein the genetic molecule payload is electrostatically encapsulated with the gelatin and covalently conjugated to the surface of the nanoparticle.

11. The genetic delivery nanoparticle of claim 10, wherein the materials attached to the surface include an antibody.

12. A method for forming genetic delivery nanoparticle, comprising:
forming an adduct of gelatin, the lipidoid and a genetic payload; and
cross-linking the gelatin to form the genetic delivery nanoparticle.

13. The method of claim 12, wherein the lipidoid comprises Go-Cx.

14. The method of claim 13, wherein x=8 to 14.

15. The method of claim 12, wherein the genetic molecule payload comprises an RNA, DNA or Crispr system payload.

16. The method of claim 12, comprising attaching genetic molecule payload to a surface of the genetic delivery nanoparticle.

17. The method of claim 12, wherein forming an adduct comprises adding a lipidoid-genetic molecule complex into a gelatin solution, adding warm ethanol while spinning the solution to form nanoparticles, cross-linking the nanoparticles with glutaraldehyde.

18. The method of claim 17, comprising conjugating antibody and PEG to a surface of the nanoparticle.

19. The method of claim 17, comprising conjugating genetic molecule on the surface of the nanoparticle.

20. The method of claim 17, wherein the lipidoid-genetic molecule complex is added dropwise into the gelatin solution.
